(19) United States
(12) Patent Application Publication
(10) Pub. No.: US 2012/0232173 A1
(43) Pub. Date: Sep. 13, 2012

(54) High Energy Power Plant Fuel, and CO or CO2 Sequestering Process

(76) Inventors: James Charles Juranitch, Ft. Lauderdale, FL (US); Thomas R. Juranitch, DelRay Beach, FL (US)

(21) Appl. No.: 13/382,155

(22) PCT Filed: Jul. 2, 2010
(86) PCT No.: PCT/US10/01930

§ 371 Date: May 29, 2012

Related U.S. Application Data
(63) Continuation-in-part of application No. PCT/US2009/003934, filed on Jul. 1, 2009.
(60) Provisional application No. 61/270,035, filed on Jul. 3, 2009.
(60) Provisional application No. 61/281,668, filed on Nov. 19, 2009.

(51) Int. Cl. C07C 27/00; C12M 1/00; B01J 19/30; F01N 3/10; B01J 19/00

(52) U.S. Cl. 518/704; 422/168; 422/129; 422/600; 422/139; 422/629; 435/289.1; 422/198

(57) ABSTRACT

A system for producing a high hydrogen to carbon ratio fuel centered approximately around C9 treats an exhaust stream from a manufacturing plant processes. The exhaust stream is processed in a Fischer Tropsch reactor, and contains CO and/or CO₂, which is sequestered, and can be a full stack exhaust stream. The Fischer Tropsch reactor is a pellet style reactor, a foam reactor, or an alpha alumina oxide foam reactor. A plasma chamber generates H₂ for reacting in the Fischer Tropsch reactor. A portion of the exhaust stream is consumed in the plasma chamber. An algae reactor converts sequestered CO₂ to O₂. The algae is exposed to the exhaust stream to extract nutrients therefrom and augment its growth. The plasma chamber receives at a high temperature region thereof CO or CO₂ that is reduced to its elemental state. The product stream and fuel are condensed and separated, and re-burned as fuel.

### RELATIONSHIP TO OTHER APPLICATIONS

This application claims the benefit of the filing date of U.S. Provisional Patent Application Ser. No. 61/281,668, filed Nov. 19, 2009, Confirmation No. 5332 (Foreign Filing License Granted); and of U.S. Provisional Patent Application Ser. No. 61/270,035, filed Jul. 3, 2009, Confirmation No. 9380 (Foreign Filing License Granted); and is a continuation-in-part of copending International Patent Application Serial Number PCT/US2009/003934, filed Jul. 1, 2009, which claims the benefit of the filing date of U.S. Provisional Patent Application Ser. No. 61/133,596, filed Jul. 1, 2008; and which further claims the benefit of the filing dates of, U.S. Provisional Patent Application Ser. Nos. 61/199,837, filed Nov. 19, 2008; 61/199,761 filed Nov. 19, 2008; 61/201,464, filed Dec. 10, 2008; 61/199,760, filed Nov. 19, 2008; 61/199,828 filed Nov. 19, 2008, and 61/208,483, filed Feb. 24, 2009; the disclosures of all of which are incorporated herein by reference.

### BACKGROUND OF THE INVENTION

1. Field of the Invention

This invention relates generally to a system for creating a high energy density, clean burning fuel as its own process or with the additional benefit of treating the exhaust output of a power plant or other CO or CO₂ liberating industrial process at the same time. In this invention a high energy density, renewable fuel is also produced when carbon neutral or carbon negative feed stocks such as municipal solid waste, biomass and/or algae are used to reduce greenhouse gas emissions into the atmosphere.

2. Description of the Prior Art

The world is concerned with global climate change. Previously this was called “global warming” but current thought directs one to think of it more as a global climate change. Many feel man, and more specifically greenhouse gasses, are responsible for a significant part of global climate change.

There is a need for a CO₂ sequestering system, or a renewable energy generating system, that is energy efficient, more cost effective, and smaller in size, than conventional systems for treating a renewable or other reactant, an exhaust stream from a power plant, or other manufacturing process. The present invention fulfils that need and produces a valuable fuel in the same process.

### SUMMARY OF THE INVENTION

In accordance with a first method aspect of the invention, there is provided a method of manufacturing a fuel on a large scale. In an advantageous embodiment of this method aspect of the invention, the fuel can be centered with an average carbon count of approximately C9 and a hydrogen ratio of approximately 3. The method includes the steps of:

supplying a waste material to a plasma melter;

supplying electrical energy to the plasma melter;

supplying water to the plasma melter;

extracting a syngas from the plasma melter;

extracting hydrogen from the syngas; and

forming fuel from the hydrogen produced in the step of extracting hydrogen.

In one embodiment, the step of supplying water to the plasma melter includes the step of supplying steam to the plasma melter. The step of supplying a waste material to the plasma melter includes the step of supplying municipal waste to the plasma melter. Also, the step of supplying a waste material to the plasma melter includes the step of supplying municipal solid waste to the plasma melter, and the step of supplying a waste material to the plasma melter includes the step of supplying a biomass to the plasma melter, the biomass being grown specifically for the purpose of being supplied to a plasma melter, and in some embodiments is algae.

In a still further embodiment of the invention, the step of extracting hydrogen from the syngas includes the steps of subjecting the syngas to a water gas shift process to form a mixture of hydrogen and carbon dioxide, and extracting hydrogen from the mixture of hydrogen and carbon dioxide. The step of extracting hydrogen from the mixture of hydrogen and carbon dioxide includes, in some embodiments, the step of subjecting the mixture of hydrogen and carbon dioxide mixture to a pressure swing adsorption process. In some embodiments, the step of extracting hydrogen from the mixture of hydrogen and carbon dioxide includes the step of subjecting the mixture of hydrogen and carbon dioxide mixture to a molecular sieve, or membrane. Also, the step of extracting hydrogen from the mixture of hydrogen and carbon dioxide includes the step of subjecting the mixture of hydrogen and carbon dioxide to an aqueous ethanolamine solution. In still further embodiments, prior to performing the step of subjecting the syngas to a water gas shift process to form a mixture of hydrogen and carbon dioxide there is provided the step of pretreating the output of the plasma melter to perform a cleaning of the syngas. Additionally, prior to performing the step of subjecting the syngas to a water gas shift process to form a mixture of hydrogen and carbon dioxide there is provided, in some embodiments of the invention, the step of pretreating the output of the plasma melter to perform a separation of the syngas.

In a further embodiment of the invention, the step of forming fuel from the hydrogen produced in the step of extracting hydrogen includes the step of subjecting the hydrogen to a pellet style Fischer Tropsch catalytic process. Prior to performing the step of forming fuel from the hydrogen produced in the step of extracting hydrogen there is provided the further step of optimizing the production of fuel by correcting the molar ratio of carbon monoxide and hydrogen in the Fischer Tropsch catalytic process. Moreover, the step of correcting the molar ratio of carbon monoxide and hydrogen in the Fischer Tropsch catalytic process includes the step of supplying a mixture of hydrogen and carbon monoxide to the Fischer Tropsch catalytic process. This step includes, in some embodiments. the step of diverting a portion of the hydrogen and carbon monoxide produced by the plasma melter, this step being performed after performing a step of cleaning the hydrogen and carbon monoxide produced by the plasma melter.

In a further embodiment of the invention, there is further provided the step of extracting a slag from the plasma melter. The plasma melter is operated in a pyrolysis mode.

In accordance with a system aspect of the invention, there is provided a system for treating an exhaust stream issued by a power plant, the system comprising the step of processing the exhaust stream in a Fischer Tropsch catalyst reactor optimized to produce a fuel of approximately C9 on average with a hydrogen ratio of approximately 3. In respective embodiments of the invention, the exhaust stream contains CO or CO₂. Additionally, the exhaust stream is, in some embodiments, a full stack exhaust stream. The Fischer Tropsch catalyst reactor is, in some embodiments, a pellet style of methanol reactor that is a foam reactor, or an alpha alumina oxide foam reactor.

There is additionally provided in some embodiments of the invention a plasma chamber for generating H₂ for reacting in the methanol reactor. A portion of the exhaust stream issued by the power plant is consumed in the plasma chamber. In further embodiments, there is provided a fluidized bed for generating H₂. A steam process is employed in some embodiments for generating H₂, and there is provided a steam reformation process in some such embodiments for generating H₂. A secondary steam reformation process that is powered by the sensible heat in a plasma exhaust is used in some embodiments to generate additional amounts of H₂.

A hydrolysis process is employed in some embodiments of the invention for generating H₂. In further embodiments, there is further provided an algae reactor for converting sequestered CO₂ to O₂. Algae is exposed to the exhaust stream of the power plant to extract nutrients from the exhaust stream to augment the growth of the algae.

In some embodiments, a plasma chamber receives at a high temperature region thereof CO that is reduced to its elemental state. In further embodiments, the exhaust stream and methanol are cooled to a temperature under 65° C. to cause liquid fuel to precipitate out. The fuel is re-burned as an energy source.

In accordance with a further system aspect of the invention, there is provided a system for treating an exhaust stream issued by a power plant. The system includes a plasma chamber for receiving at a high temperature region thereof CO that is reduced to its elemental state.

In a method aspect of a specific illustrative embodiment of the invention, there is provided the step of processing the feedstock and exhaust stream in a pellet style, foam style, or alpha alumina oxide foam style, Fischer Tropsch catalyst. The catalyst has been developed with a specific alpha and operating condition that centers it product output around the C9 value. This advantageous design can be leveraged in its high condensing temperature, especially when combined with the advantageous high flow, high conversion, properties of a foam Fischer Tropsch catalyst. On average a C9 compound will condense at 126° C. This high temperature allows this process to capture CO or CO₂ in an energy efficient way. The CH ratio is also approximately 1:3.4 which makes for a very clean burning fuel.

This invention is directed generally to an efficient method of, and system for, sequestering CO₂ and/or CO from a process or an exhaust stream. The CO or CO₂ is then converted to a high energy density fuel currently and used as a transportable fuel, or burned in the manufacturing process that required heat. When carbon neutral or carbon negative feed stocks such as biomass, municipal solid waste, and algae are used, green house gas emissions into the atmosphere are significantly reduced.

In a further embodiment, there is provided a plasma chamber for receiving at a high temperature region thereof CO₂ that is thereby shifted or reduced

### BRIEF DESCRIPTION OF THE DRAWING

Comprehension of the invention is facilitated by reading the following detailed description, in conjunction with the annexed drawing, in which:

FIG. 1 is a simplified schematic representation of a plurality of power plants and industrial processes issuing greenhouse gas exhaust that is treated in a modified Fischer Tropsch reactor and a fuel condensate system;

FIG. 2 is a simplified schematic representation of a further embodiment of the system shown in FIG. 1, wherein a plurality of power plants and industrial processes issue greenhouse gas exhaust that is treated in a Fischer Tropsch reactor and a fuel condensate system; and

FIG. 3 is a simplified schematic representation of a fuel manufacturing system that does not use an industrial exhaust stream as a feed stock.

### DETAILED DESCRIPTION

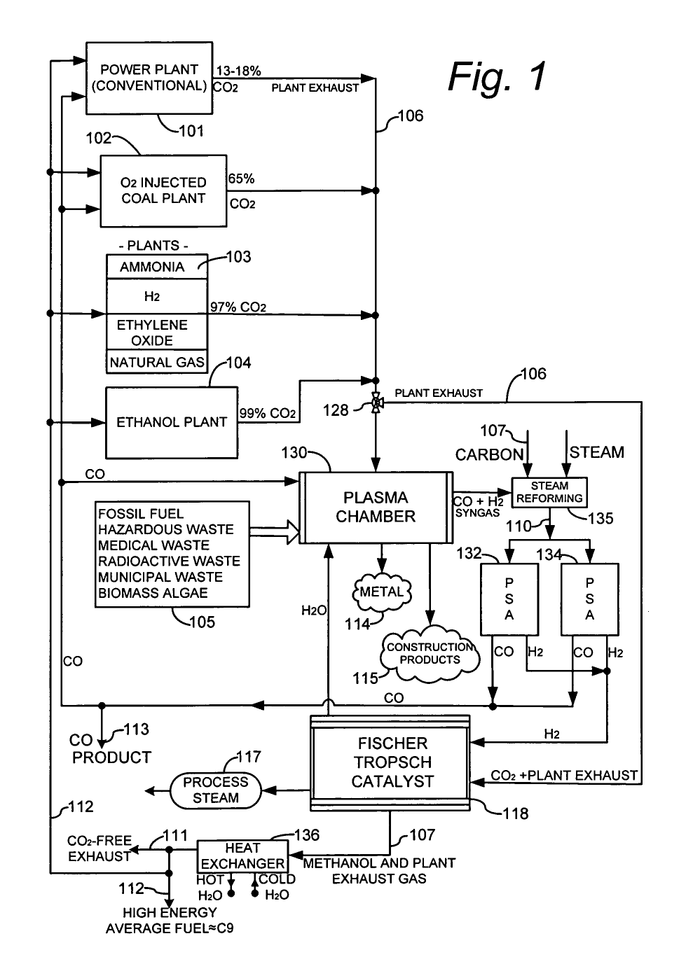

FIG. 1 shows a number of plants, specifically conventional power plant 101, O₂ injected coal plant 102, plants 103 (ammonia, H₂, ethylene oxide, and natural gas) that produce CO₂. Coal fired conventional power plant 101 emits about two pounds of CO₂ per kiloWatt-hour (“kW-h”). A cleaner competitor is a conventional natural gas power plant. It would look substantially the same as the conventional coal fired power plant, yet would emit only about 1.3 pounds of CO₂ per kW-h. All such plants are significant contributors to the global inventory of greenhouse gasses.

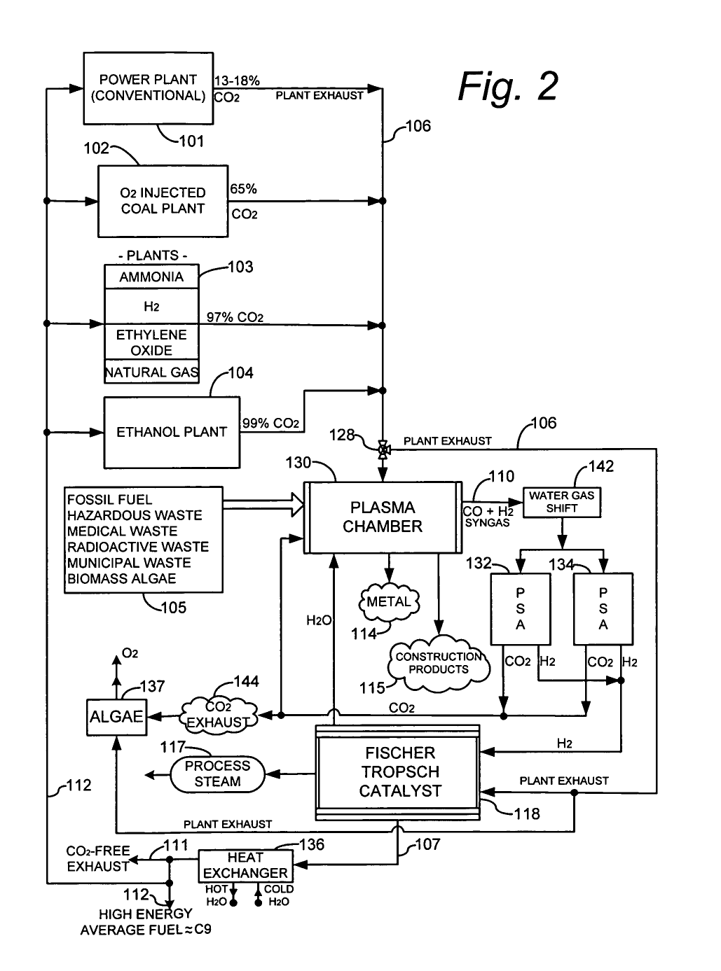

Plants 102, 103, and 104 illustrate increasing concentrations of CO₂ per plant exhaust volume. However, the low ratio of CO₂ per exhaust volume issued by power plant 101 renders sequestration of CO₂ expensive and difficult. Some power plant systems have been demonstrated as able to achieve less expensive and less difficult CO₂ sequestration, but they are capital and energy intensive. After the CO or CO₂ is sequestered it still has to be stored in a conventional sequestering system (not shown). Moreover, the storage of CO₂ is expensive and controversial. However, the present invention enables the processing of CO₂ on site, and the storage thereof is not necessary. This is particularly feasible when carbon neutral, or carbon negative, feed stocks are used, such as algae. Post processing of the CO₂ in an algae reactor, such as algae reactor 137 (FIG. 2) enables carbon negative operation.

Referring once again to FIG. 1, plant exhaust stream 106 is delivered to a plasma chamber 130 and then to a Fischer Tropsch reactor 118. A small percentage of the flow is typically fed into plasma reactor 130. Fischer Tropsch reactor 118 is, in some embodiments of the invention, a foam, or alumina oxide foam reactor, but can be any composition that converts CO₂ into a carbon chain of approximately C9 on average. Plasma chamber 130 is used as a hydrogen generator. In the practice of the invention, any suitable hydrogen generator can be used. However, in the present state of the art a plasma reactor is one of the most efficient, and therefore is shown in this embodiment of the invention. In other embodiments, a conventional gassifier (not shown) or fluidized bed (not shown) can also be used.

Plasma chamber 130 can be supplied from any of several feed stocks 105. These include a fossil fuel such as coal, hazardous waste, medical waste radioactive waste, municipal waste, or a carbon negative fuel such as algae. The plasma chamber will exhausts a product gas that consists primarily of syngas at a temperature, in this specific illustrative embodiment of the invention, of approximately 1200° C. This flow contains considerable sensible heat energy that is to be extracted at flow stream 110 to make carbon efficient electrical or steam power. A steam reforming process 135 is operated in the specific illustrative embodiment of the invention shown in FIG. 1 directly in the high temperature plasma flow stream, or indirectly in a closed loop heat transfer system to generate additional H₂.

Carbon, which is provided at carbon inlet 107, is obtained from conventional sources such as methane (not shown), or from unconventional sources such as semi-spent fly ash (not shown). Syngas 110 then is processed through pressure swing absorbers 132 and 134 to separate the H₂ from the CO. In the practice of the invention, any conventional form of separation system, such as membranes/molecular sieves, (not shown), aqueous solutions (not shown), Pressure swing adsorber, (not shown), etc. can be used in other embodiments of the invention to separate out the H₂. The H₂ then is delivered to Fischer Tropsch catalyst reactor 118 where it is in this embodiment combined with plant exhaust flow 106.

Fischer Tropsch catalyst reactor 118 can, in respective embodiments of the invention, be a conventional reactor or it can be a foam reactor or an alpha alumina oxide foam reactor in an idealized application. Alpha alumina oxide foam reactors accommodate a considerably larger flow rate than conventional reactors, such increased flow being advantageous in the practice of the invention.

Plant exhaust 106 and H₂ react exothermically in Fischer Tropsch catalyst reactor 118. The resulting heat is, in this embodiment of the invention, extracted as steam 117 that can be used in numerous parts of the process herein disclosed, such as in plasma reactor 130 (connection for delivery not shown), steam reformation chamber 135 (connection for delivery not shown), or as municipal steam. The combined fuel and exhaust gas at Fischer Tropsch catalyst reactor outlet 107 are then delivered, in this embodiment, to heat exchanger 136. Using cold water in this embodiment, heat exchanger 136 brings the temperature of the gaseous mixture below 65° C., which precipitates out the product fuel in a liquid form at liquid high energy fuel outlet 112 at a pressure of one atmosphere. The liquid fuel at outlet 112 is separated from the CO and or CO₂ depleted plant exhaust which then, in this specific illustrative embodiment of the invention, is exhausted to the atmosphere from CO₂-reduced exhaust outlet 111. The liquid high energy fuel can be sold to, or recycled into, any of the plants to produce heat.

The CO from the syngas, which is available in this embodiment of the invention at CO product outlet 113, can be sold as a product, or in some embodiments of the invention, be reintroduced into plasma chamber 130 at the high temperature zone thereof (not shown), which can operate at approximately 7000° C., to be reduced into elemental forms of carbon and oxygen. This process can be aided, in some embodiments, by microwave energy, magnetic plasma shaping, UHF energy, corona discharge, or laser energy (not shown). Additionally, the CO can be reintroduced into the plant to be burned as fuel that yields approximately 323 BTU/cu ft.

FIG. 2 is a simplified schematic representation of a further embodiment of the system shown in FIG. 1, wherein a plurality of power plants issue greenhouse gas exhaust that is treated in a Fischer Tropsch catalyst reactor and a fuel condensate system. Elements of structure that have previously been discussed are similarly designated. In this figure, there is shown a further example of the process wherein there is provided a gas shift reaction 142 that is disposed downstream of the syngas generating plasma chamber 130. A steam reformation system 135 (FIG. 1) can optionally be employed in the embodiment of FIG. 2. The CO₂ that has been separated by operation of Pressure swing adsorbers 132 and 134 is, in this embodiment of the invention, processed by an algae reactor 137. Algae reactor 137 is, in some embodiments, a photoreactor or a hybrid pond. In addition, a portion of plant exhaust 106 is processed by the algae to provide growth accelerating elements such as nitrogen. Any conventional process other than Pressure swing adsorbers can be used in other embodiments of the invention to separate the CO₂ from the shifted syngas.

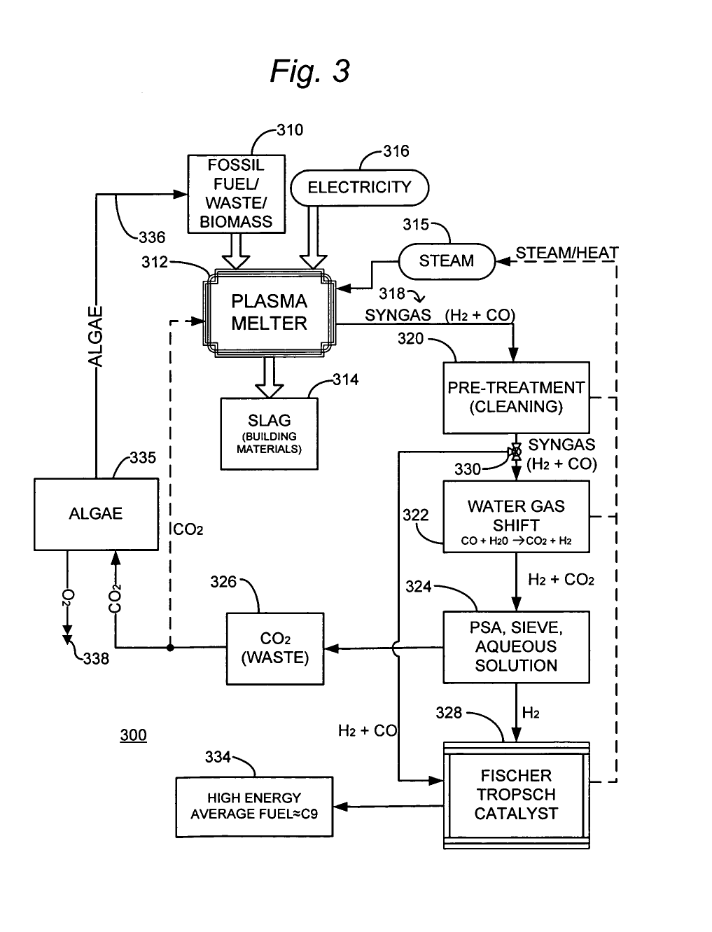

In some cases the high energy fuel maybe desired to be made at a remote location without access to a plant exhaust stream and then transported to a plant for consumption. An example of this is shown in FIG. 3. The present invention is particularly relevant if a combination of biomass, municipal solid waste, or other renewable groups of feedstocks are used. This will allow the plant that consumes the fuel to claim a percentage of renewable credits per fuel burned. The exhaust will also be credited with the appropriate amount of carbon neutral credits. In this case the foregoing and other objects are achieved by this invention which includes the steps of:

supplying a waste material to a plasma melter;

supplying electrical energy to the plasma melter;

supplying water to the plasma melter;

extracting a syngas from the plasma melter;

extracting hydrogen from the syngas; and

forming a high hydrogen/carbon ratio fuel centered at approximately C9 from the hydrogen produced in the step of extracting hydrogen.

In one embodiment of the invention, the step of supplying water to the plasma melter comprises the step of supplying steam to the plasma melter.

In an advantageous embodiment of the invention, the waste material that is supplied to the plasma melter is a municipal waste. In other embodiments, the waste material is a municipal solid waste, and in still other embodiments the waste material is a biomass. In some embodiments where the waste material is a biomass, the biomass is specifically grown.

In one embodiment of the invention, the step of extracting hydrogen from the syngas includes, but is not limited to, the steps of:

subjecting the syngas to a water gas shift process to form a mixture of hydrogen and carbon dioxide; and

directing a portion of the CO₂ flow to an algae bioreactor or pond or to be reprocessed in the plasma chamber.

The water gas shift process is primarily used to extract additional hydrogen from the product mixture of hydrogen and carbon dioxide.

In a further embodiment, the step of extracting hydrogen from the mixture of hydrogen and carbon dioxide includes, but is not limited to, the step of subjecting the mixture of hydrogen and carbon dioxide mixture to a pressure swing adsorption process. In some embodiments, the step of extracting hydrogen from the mixture of hydrogen and carbon dioxide includes, but is not limited to, the step of subjecting the mixture of hydrogen and carbon dioxide mixture to a molecular sieve or membrane. In a further embodiment, the step of extracting hydrogen from the mixture of hydrogen and carbon dioxide includes, but is not limited to, the step of subjecting the mixture of hydrogen and carbon dioxide mixture to an aqueous ethanolamine solution. In yet another embodiment, prior to performing the step of subjecting the syngas to a water gas shift process to form a mixture of hydrogen and carbon dioxide there is provided the step of pre treating the output of the plasma melter to perform a cleaning and separation of the syngas.

In accordance with an advantageous embodiment of the invention, the step of forming the product fuel from the hydrogen produced in the step of extracting hydrogen includes, without limitation, the step of subjecting the hydrogen to a Fischer Tropsch catalytic process. In one embodiment, prior to performing the step of forming a fuel from the hydrogen produced in the step of extracting hydrogen there is provided the further step of optimizing the production of the fuel by correcting the molar ratio of CO and hydrogen in the Fischer Tropsch catalytic process. The step of correcting the molar ratio of CO and hydrogen in the Fischer Tropsch catalytic process includes, but is not limited to, the step of supplying a mixture of hydrogen and carbon monoxide to the Fischer Tropsch catalytic process.

In an advantageous embodiment of the invention, the step of supplying the mixture of hydrogen and carbon monoxide to the Fischer Tropsch process includes, but is not limited to, the step of diverting a portion of the hydrogen and carbon monoxide produced by the plasma melter. The step of diverting a portion of the hydrogen and carbon monoxide produced by the plasma melter is performed, in one embodiment, after performing a step of cleaning the hydrogen and carbon monoxide produced by the plasma melter.

In an advantageous embodiment of the invention, there is provided the step of extracting a slag from the plasma melter. In a further embodiment, the step of supplying a waste material to the plasma melter includes, but is not limited to, the step of supplying municipal waste to the plasma melter.

FIG. 3 is a simplified function block and schematic representation of a specific illustrative embodiment of the invention. As shown in this figure, a fuel producing system 300 receives fossil fuel, municipal waste, or specifically grown biomass 310 that is deposited into a plasma melter 312. In the practice of some embodiments of the invention, the process is operated in a pyrolysis mode (i.e., lacking oxygen). Water, which in this specific illustrative embodiment of the invention is used in the form of steam 315, is delivered to plasma melter 312 to facilitate production of hydrogen and plasma. Also, electrical power 316 is delivered to plasma melter 312. A hydrogen rich syngas 318 is produced at an output (not specifically designated) of plasma melter 312, as is a slag 314 that is subsequently removed.

In some applications of the invention, slag 314 is sold as building materials, and may take the form of mineral wool, reclaimed metals, and silicates, such as building blocks. In some embodiments of the invention, the BTU content, plasma production, and slag production can also be “sweetened” by the addition of small amounts of coke or other additives (not shown).

The syngas is cooled and cleaned, and may be separated in certain embodiments of the invention, in a pretreatment step 320. The CO is processed out of the cleaned syngas at the output of a Water Gas Shift reaction 322. The waste carbon dioxide 326 that is later stripped out may not be considered an addition to the green house gas carbon base. This would be due to the fact it could be obtained in its entirety from a reclaimed and renewable source energy. For example in this embodiment of the invention, the energy source could be predominantly municipal waste 310.

In some embodiments, the carbon dioxide is recycled into the plasma melter 312 and reprocessed into CO and hydrogen. A Pressure Swing Adsorption process, molecular sieve/membrane, aqueous ethanolamine solutions, or other processes are used in process step 324 to separate out carbon dioxide 326. A portion of this carbon dioxide can be directed to a algae bioreactor 335 or redirected to the plasma melter 310 for reprocessing. The algae can be used again as a feedstock for the plasma converter 310. Hydrogen from process step 324 is delivered to the optimized Fischer Tropsch Catalyst process 328.

In this specific illustrative embodiment of the invention, a portion of the CO and hydrogen obtained from pretreatment step 320 is diverted by a flow control valve 330 and supplied to the Fischer Tropsch Catalyst process 328. This diverted flow is applied to achieve an appropriate molar ratio of CO and hydrogen, and thereby optimize the production of fuel.

Pretreatment step 320, Water Gas Shift reaction 322, and Fischer Tropsch Catalyst process 328 generate heat that in some embodiments of the invention is used to supply steam to the plasma melter 312, or to a turbine generator (not shown), or any other process (not shown) that utilizes heat.

Although the invention has been described in terms of specific embodiments and applications, persons skilled in the art may, in light of this teaching, generate additional embodiments without exceeding the scope or departing from the spirit of the invention herein claimed. Accordingly, it is to be understood that the drawing and description in this disclosure are proffered to facilitate comprehension of the invention, and should not be construed to limit the scope thereof.

What is claimed is:

1. A method of manufacturing a fuel on a large scale, the fuel is centered with an average carbon count of approximately C9 and a hydrogen ratio of approximately 3. the method having the steps of:
supplying a waste material to a plasma melter;
supplying electrical energy to the plasma melter;
supplying water to the plasma melter;
extracting a syngas from the plasma melter;
extracting hydrogen from the syngas; and
forming fuel from the hydrogen produced in said step of extracting hydrogen.

2. The method of claim 1, wherein said step of supplying water to the plasma melter comprises the step of supplying steam to the plasma melter.

3. The method of claim 1, wherein said step of supplying a waste material to the plasma melter comprises the step of supplying municipal waste to the plasma melter.

4. The method of claim 1, wherein said step of supplying a waste material to the plasma melter comprises the step of supplying municipal solid waste to the plasma melter.

5. The method of claim 1, wherein said step of supplying a waste material to the plasma melter comprises the step of supplying a biomass to the plasma melter.

6. The method of claim 5, wherein the biomass is specifically grown for being supplied to a plasma melter such as algae.

7. The method of claim 1, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

8. The method of claim 7, wherein said step of extracting hydrogen from the mixture of hydrogen and carbon dioxide comprises the step of subjecting the mixture of hydrogen and carbon dioxide mixture to a pressure swing adsorption process.

9. The method of claim 7, wherein said step of extracting hydrogen from the mixture of hydrogen and carbon dioxide comprises the step of subjecting the mixture of hydrogen and carbon dioxide mixture to a molecular sieve, or membrane.

10. The method of claim 7, wherein said step of extracting hydrogen from the mixture of hydrogen and carbon dioxide comprises the step of subjecting the mixture of hydrogen and carbon dioxide to an aqueous ethanolamine solution.

11. The method of claim 7, wherein prior to performing said step of subjecting the syngas to a water gas shift process to form a mixture of hydrogen and carbon dioxide there is provided the step of pre treating the output of the plasma melter to perform a cleaning of the syngas.

12. The method of claim 7, wherein prior to performing said step of subjecting the syngas to a water gas shift process to form a mixture of hydrogen and carbon dioxide there is provided the step of pre treating the output of the plasma melter to perform a separation of the syngas.

13. The method of claim 1, wherein said step of forming fuel from the hydrogen produced in said step of extracting hydrogen comprises the step of subjecting the hydrogen to a pellet style Fischer Tropsch catalytic process.

14. The method of claim 13, wherein prior to performing said step of forming fuel from the hydrogen produced in said step of extracting hydrogen there is provided the further step of optimizing the production of fuel by correcting the molar ratio of carbon monoxide and hydrogen in the Fischer Tropsch catalytic process.

15. The method of claim 14, wherein said step of correcting the molar ratio of carbon monoxide and hydrogen in the Fischer Tropsch catalytic process comprises the step of supplying a mixture of hydrogen and carbon monoxide to the Fischer Tropsch catalytic process.

16. The method of claim 15, wherein said step of supplying the mixture of hydrogen and carbon monoxide to the Fischer Tropsch process comprises the step of diverting a portion of the hydrogen and carbon monoxide produced by the plasma melter.

17. The method of claim 16, wherein said step of diverting a portion of the hydrogen and carbon monoxide produced by the plasma melter is performed after performing a step of cleaning the hydrogen and carbon monoxide produced by the plasma melter.

18. The method of claim 1, wherein there is further provided the step of extracting a slag from the plasma melter.

19. The method of claim 1, wherein the plasma melter is operated in a pyrolysis mode.

20. The method of claim 1, wherein said step of forming fuel from the hydrogen produced in said step of extracting hydrogen comprises the step of subjecting the hydrogen to a alpha alumina oxide foam style Fischer Tropsch catalytic process.

21. The method of claim 1, wherein said step of forming fuel from the hydrogen produced in said step of extracting hydrogen comprises the step of subjecting the hydrogen to a foam style Fischer Tropsch catalytic process.

22. A system for treating an exhaust stream issued by a power plant, the system comprising the step of processing the exhaust stream in a Fischer Tropsch catalyst reactor optimized to produce a fuel of approximately C9 on average with a hydrogen ratio of approximately 3.

23. The system of claim 22, wherein the exhaust stream contains CO.

24. The system of claim 22, wherein the exhaust stream contains CO₂.

25. The system of claim 22, wherein the exhaust stream is a full stack exhaust stream.

26. The system of claim 22, wherein the Fischer Tropsch catalyst reactor is a pellet style of methanol reactor.

27. The system of claim 22, wherein the methanol reactor is a foam reactor, or an alpha alumina oxide foam reactor.

28. The system of claim 22, wherein there is further provided a plasma chamber for generating H₂ for reacting in the methanol reactor.

29. The system of claim 28, wherein a portion of the exhaust stream issued by the power plant is consumed in the plasma chamber.

30. The system of claim 22, wherein there is further provided a fluidized bed for generating H₂.

31. The system of claim 22, wherein there is further provided a steam process for generating H₂.

32. The system of claim 22, wherein there is further provided a steam reformation process for generating H₂.

33. The system of claim 32, wherein there is further provided a secondary steam reformation process that is powered by the sensible heat in a plasma exhaust, for generating additional amounts of H₂.

34. The system of claim 22, wherein there is further provided a hydrolysis process for generating H₂.

35. The system of claim 22, wherein there is further provided an algae reactor for converting sequestered CO₂ to O₂.

36. The system of claim 22, wherein algae is exposed to the exhaust stream of the power plant to extract nutrients from the exhaust stream to augment the growth of the algae.

37. The system of claim 22, wherein there is further provided a plasma chamber for receiving at a high temperature region thereof CO that is reduced to its elemental state.

38. The system of claim 22, wherein the exhaust stream and methanol are cooled to a temperature under 65° C. to cause liquid fuel to precipitate out.

39. The system of claim 22, wherein the fuel is re burned as an energy source.

40. A system for treating an exhaust stream issued by a power plant, the system comprising a plasma chamber for receiving at a high temperature region thereof CO that is reduced to its elemental state.
